(19) United States
(12) Patent Application Publication
(10) Pub. No.: US 2026/0275418 A1
(43) Pub. Date: Sep. 17, 2026

(54) SCHIZOPHRENIA BIOMARKER AND METHOD OF DIAGNOSIS AND KIT OF PARTS

(71) Applicants: UNIVERSIDAD ANDRÉS BELLO, Santiago (CL); UNIVERSIDAD DE CHILE, Comuna de Santiago (CL)

(72) Inventors: Bárbara Sofía CASAS ATALA, Santiago (CL); Elvis ACEVEDO RIQUELME, Santiago (CL); Martín Alejandro MONTECINO LEONARD, Santiago (CL); Verónica Alejandra PALMA ALVARADO, Santiago (CL)

(21) Appl. No.: 19/403,582

(22) Filed: Nov. 28, 2025

Related U.S. Application Data
(60) Provisional application No. 63/769,879, filed on Mar. 11, 2025.

(51) Int. Cl. C12Q 1/70; C12Q 1/6809; C12Q 1/6881; C12Q 1/6883; C12Q 1/6886

(52) U.S. Cl. CPC C12Q 1/6883; C12Q 1/6809; C12Q 1/6881; C12Q 1/6886; C12Q 1/701; C12Q 2600/112; C12Q 2600/154; C12Q 2600/156; C12Q 2600/158; G01N 2800/302

(57) ABSTRACT

A method is disclosed for detecting schizophrenia, at any stage, which comprises the steps of determining the gene expression of ZNF560 in a biological sample from a subject and comparing it with the ones of a healthy individual as a control sample, wherein the subject of the biological sample is diagnosed as having schizophrenia the gene ZNF560 is overexpressed in the biological sample in comparison to the control. Also disclosed is a kit of parts to diagnose schizophrenia from a biological sample and the use of the kit of parts as a detection agent useful to diagnose schizophrenia, wherein schizophrenia could be at prodromal, acute, residual, or chronic stage.

### REFERENCE TO SEQUENCE LISTING SUBMITTED ELECTRONICALLY

The sequence listing of the present application is submitted electronically via Patent Center as an ST.26 XML formatted sequence listing with the file name “81878US_Sequence_listing.xml”, with a creation date of Nov. 17, 2025, and a file size of 13,299 bytes. This sequence listing is incorporated herein by reference in its entirety.

### BACKGROUND OF THE INVENTION

Schizophrenia is one of the most severe and disabling mental disorders. It is a chronic mental disorder characterized by perturbations in thinking, perception, and behavior, impairing people's ability to lead an independent life. It is considered a disease with a high social and economic burden, both for individuals and for public health systems. Particularly, it is estimated to affect between 0.3 and 1% of the world population (OPS, 20232; Stepnicki et al., 2018), depending on the region.

In Chile, according to the Ministry of Health (MINSAL), prevalence is estimated to be close to 1%. Furthermore, according to the Chilean National Mental Health Plan, psychiatric illnesses account for 25% of the total disease burden in the country, with schizophrenia being one of the main causes of disability among young adults (15-44 years). Chilean men present an average onset of schizophrenia at 20-25 years of age and women at 25-30 years, similar to international trends (MINSAL). Since 2005, the diagnosis and treatment of schizophrenia have been covered by the Law of Explicit Health Guarantees (GES), and both the current legal framework and ministerial guidelines promote a community-based model, with a rights-based approach and comprehensive, person-centered care.

However, diagnosis continues to be a problem, because it is based on a standardization of symptomatology during the acute phase and does not take into account other elements that are important for the patient.

On average, the diagnosis of schizophrenia occurs between 1 and 2 years after the onset of psychotic symptoms (OPS, 2023. During this “period of untreated psychosis” (DUP), the progression of neurological damage is more severe, symptoms become chronic, and the response to subsequent treatments is poorer.

The diagnosis of schizophrenia is currently based exclusively on the clinical evaluation of symptoms, since there are no validated laboratory or imaging tests to confirm it. Unlike neurodegenerative disorders such as Alzheimer's disease, which exhibit defined neuropathological lesions, schizophrenia does not show specific macroscopic brain alterations, but rather subtle changes distributed across multiple regions. For this reason, there is interest in diagnoses based on molecular, genetic or cellular biological markers, in order to develop specific methodologies to detect this disease. For example, patent CN113667734B protects a diagnostic kit for schizophrenia based on the detection of methylation in a sequence of the SHANK3 gene. However, detection is carried out on differentiated neurons, which is a lengthy and costly method to perform. Similarly, patent application CN104862310A describes the use of different microRNAs as biomarkers for the diagnosis of schizophrenia. A ROC-AUC of 0.802 and a specificity of up to 82% are reported, which are rather low values to consider that a technique provides reliable specific results. Based on these shortcomings of the biomarkers currently reported for diagnosing schizophrenia, there is a need to develop a marker that allows this disease to be diagnosed rapidly, at any stage of the disorder and specifically.

Patients who are diagnosed in advanced stages have a 2.5-fold higher likelihood of prolonged hospitalizations, higher annual medical costs compared with those diagnosed early, greater functional deterioration and dependence on long-term care (Kadakia, 2022; Harrison, 2021). Moreover, schizophrenia is the costliest mental disorder per person for society (OPS, 2023).

Currently, it is recognized as a multifactorial disease where high genetic heterogeneity, environmental factors, and brain development interact to subtly alter programmed neurodevelopmental trajectories and pathways. Despite the large genetic heterogeneity identified in patients, a biological convergence phenomenon is observed at different levels, including in gene expression, molecular processes, and/or symptom appearance, which are common among many schizophrenia patients (Bimbaum & Weinberger 2017; Hoffman 2019; Singh 2022). For this reason, assessing epigenetic alterations associated with this disorder is of particular importance. Identifying differential expression of genes within healthy subjects and the ones having schizophrenia will allow determining the development of the disease.

One of the most studied epigenetic modifications in schizophrenia is DNA methylation. Differentially methylated genes in schizophrenia have been associated with neuroinflammation, metabolism, synaptic transmission, neurogenesis, and neurodevelopment. Most key biological processes are altered in this disease.

Seeking to model early brain alterations, the utilization of induced pluripotent stem cells (hiPSC) has grown importance in the schizophrenia field (Richetto 2021). Neural stem cells, neural precursors, and neurons derived from schizophrenia hiPSC show classical phenotypes of this disease, independent of the different genetic backgrounds (Karagiannis 2019; Das 2020). This observation is particularly relevant as it has been long determined that there is an erase of the epigenetic profile during reprogramming. Hence, there seems to be a convergence of phenotypes, independent of genetic background, indicating the emergence of diseases with specific epigenetic patterns during differentiation.

There is a need to identify markers that allow detecting schizophrenia, at any stage of the disorder; and to provide a method of diagnosis of schizophrenia.

### DETAILED DESCRIPTION OF THE INVENTION

The present invention is related to a biomarker for schizophrenia, corresponding to gene ZNF560, expressing the zinc finger protein 560. The inventors of the present invention have demonstrated that there is a differential expression pattern in this gene, being overexpressed in a biological sample from schizophrenia patients over healthy individuals due to a different DNA methylation profile at genomic regions that regulate ZNF560 mRNA expression. Particularly, said biomarker comprises the nucleotide sequences as shown in NCBI Accession Nos. NM_152476.3 (SEQ ID NO: 1) and NM_001351678.2 (SEQ ID NO: 2). In one embodiment, this biomarker is isolated from the genomic sequence of the individual.

The present invention is also related to a mix of primers capable of producing a cDNA of gene ZNF560. In an embodiment, the mix of primers are capable of amplifying the biomarker of gene ZNF560 as shown above, the forward primer comprises a nucleotide sequence having at least an 80%, 90% or 95% of identity of SEQ ID NO: 3 and the reverse primer comprises a nucleotide sequence having at least an 80%, 90% or 95% of identity of SEQ ID NO: 4. Preferably, the forward primer comprises a nucleotide sequence comprising SEQ ID NO: 3 and the reverse primer comprises a nucleotide sequence comprising SEQ ID NO: 4. More preferably, the forward primer comprises a nucleotide sequence consisting of SEQ ID NO: 3 and the reverse primer comprises a nucleotide sequence consisting of SEQ ID NO: 4. More preferably, the forward primer consists of a nucleotide sequence consisting of SEQ ID NO: 3 and the reverse primer consists of a nucleotide sequence consisting of SEQ ID NO: 4.

The present invention also describes a method for detecting schizophrenia, wherein the method comprises the steps of
a) providing a biological sample obtained from a subject;
b) generating hiPSC from the biological sample;
c) measuring the expression of ZNF560 gene;
d) determining the mRNA levels present in the hiPSC culture obtained from the sample; and
e) comparing these mRNA levels with the ones of a healthy individual as a control, wherein the subject of the hiPSC derived from the tested biological sample is diagnosed as having schizophrenia if mRNA levels are higher in the biological sample in comparison to the control.

Preferably, the method is in vitro or ex vivo.

In one embodiment, the sample obtained from the subject is selected from a skin biopsy, a blood sample or urine sample. Preferably, if the sample is a biopsy, this can be selected from a group consisting of skin, needle, surgical, and endoscopic biopsy, in addition to any tissue, cell or fluid from the subject.

In other embodiment, in step b), the induction or generation of hiPSC from the biological sample are protocols well-known by a person skilled in the art (Sochacki 2016, Zhou 2011, Trokovic 2014).

In another embodiment, gene expression of ZNF560 is measured by any technique that quantifies the transcription of this gene. Preferably, gene expression of ZNF560 is measured by transcriptomic analysis or an individual analysis of the gene expression. Most preferably, gene expression of ZNF560 is measured by next generation sequencing (RNA-seq) or reverse transcriptase coupled to amplification by quantitative polymerase chain reaction (RTqPCR).

In one embodiment, between steps b) and c), the method comprises the step of extracting RNA from the biological sample using any conventional and/or standard protocols or kits available in the market and known in the art.

In another embodiment, the gene expression of ZNF560 is measured by RTqPCR using any appropriate primers to generate the cDNA of gene ZNF560. Preferably, the primers have the nucleotide sequence disclosed in the Examples of the present invention.

In another embodiment, step e) comprises comparing the mRNA levels of the biological sample with the ones of a control (healthy subject) sample, wherein there are detectable levels of mRNA in a sample having overexpression of gene ZNF560, thus, a patient having schizophrenia, in comparison to the control sample, which has undetectable levels of ZNF560 transcript.

In one embodiment, said method of detection can detect schizophrenia in any stage, including prodromal, acute, residual, and chronic phases.

Moreover, the present invention refers to a kit of parts to diagnose schizophrenia from a biological sample, comprising: a) an instructions leaflet with instructions to develop hiPSC from a biological sample and to perform the technique to measure of the gene expression of ZNF560, wherein said technique is RNA-seq or a RTqPCR; and b) a mix of primers if the technique is RTqPCR, wherein these primers have the nucleotide sequences of the primers of the Examples of the present application to perform a RTqPCR. In one embodiment, the kit comprises optionally the required reagents to perform a RTqPCR, such as a fluorescent dye, a DNA polymerase, a Reverse transcriptase, dNTPs, a magnesium salt, probes and any combination thereof.

In an embodiment, the magnesium salt is selected from MgCl₂, MgSO₄ and Mg(OAc)₂. More preferably, the magnesium salt is MgCl₂. On the other hand, the present invention also discloses the use of the biomarker, the mix of primers or the kit of parts as a detection agent useful to diagnose schizophrenia, wherein schizophrenia is the prodromal, acute, residual, or chronic stage.

### DETAILED DESCRIPTION OF THE DRAWINGS

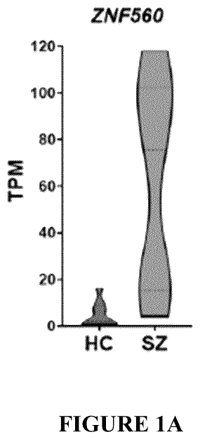

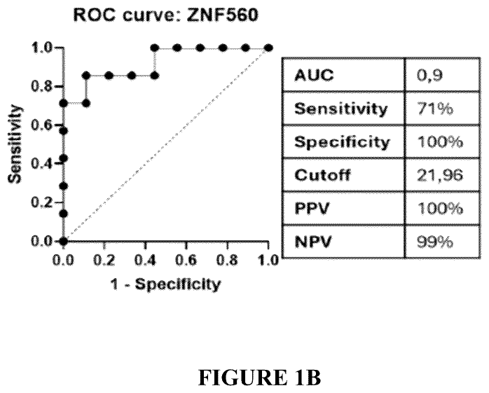

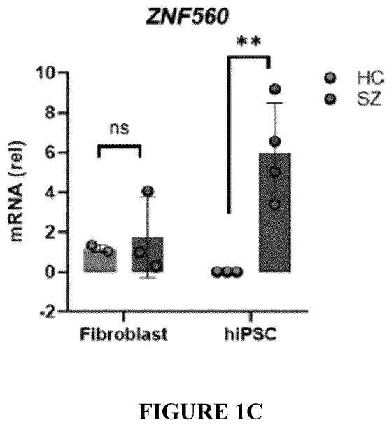

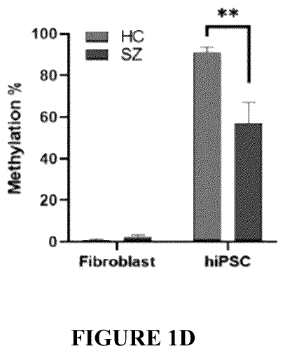

FIG. 1A-1D. ZNF560 overexpression in schizophrenia hiPSC. A) TPM values of ZNF560 for healthy control hiPSC (HC, n=10) and schizophrenia hiPSC (SZ, n=7) hiPSC, determined by RNA-seq. Graph includes all available data at GEO repository. B) ROC curve of ZNF560 TPM values. C) mRNA expression of ZNF560 in Fibroblasts and respective isogenic hiPSC from HC and SZ. D) Methylation percentage for CpG island at ZNF560 promoter in each HC or SZ fibroblast and respective isogenic hiPSC.

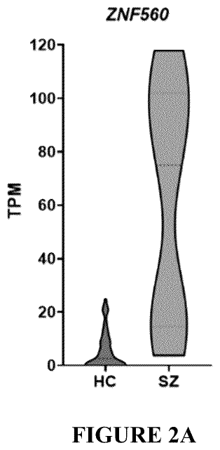

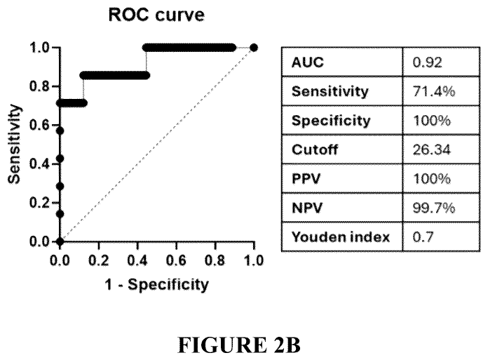

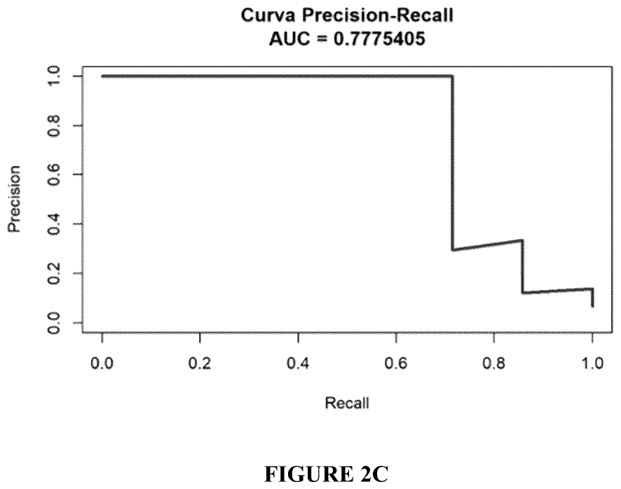

FIG. 2A-2C. Validation of RNA-seq for determining overexpression of ZNF560 between hiPSC from patients previously diagnosed with schizophrenia and healthy subjects. A) TPM values of ZNF560 for healthy control hiPSC (HC, n=9) and schizophrenia hiPSC (SZ, n=7) hiPSC. Graph includes all available data at GEO, SRA and ENA repository. B) ROC curve of ZNF560 TPM values. C) A Precision-Recall curve (PR) of ZNF560 TPM values.

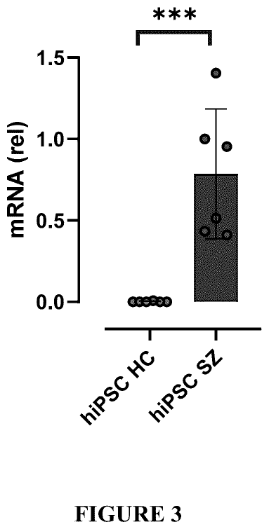

FIG. 3. Validation of qRT-PCR for determining overexpression of ZNF560 between hiPSC from patients previously diagnosed with schizophrenia and healthy subjects. ZNF560 mRNA levels relative to the expression of one hiPSC SZ sample and HC samples. The data are normalized to the expression of GAPDH. N=6 HC, N=6 SZ.

### EXAMPLES

### Example 1: Transcriptomic Analyses Using Next Generation Sequencing (RNA-Seq) at Different Stages of Neuronal Differentiation in Healthy Control and Schizophrenia hiPSC

hiPSC cells were obtained from dermal fibroblast reprogramming as described before (Sochacki 2016). Three hiPSC lines were obtained from healthy control subjects (HC hiPSC) and four from SZ patients (SZP hiPSC). Three SZP hiPSC lines were obtained from the Coriell Institute (GM23760 and GM23761) and two were reprogrammed at the D'Or Institute for Research and Education (EZQ3 and EZQ4, reported at Sochacki 2016). For the Ctrl hiPSC, a comparable age range was used as the SZP hiPSC. One Ctrl hiPSC line was obtained from the Coriell Institute (GM23279), while the other two hiPSC lines were reprogrammed at the D'Or Institute (CF1 and CF2, reported at Casas 2018). Informed consent was obtained from all donors, with the approval of the research ethics council (CAAE: 32385314.9.0000.5249)

hiPSC cell lines and transferred onto Matrigel (BD Biosciences, USA)-coated plates and cultured in mTESR1 medium (StemCells Technologies Cat Nom 85850, Canada), following manufacturer instructions.

### RNA Extraction:

Cells were homogenized in Trizol and then total RNA was extracted using Direct-zol RNA MiniPrep (Zymo Research Cat No R2052, Irvine, CA).

### RNA-Seq:

1 μg of total RNA was sent to Quick Biology (https://www.quickbiology.com/; Monrovia, CA, USA) for a standard RNA sequencing service, PolyA, 30M reads per sample, paired-end.

### Results:

The overall comparison of RNA-seq data between healthy control and schizophrenia hiPSC showed no significant difference. Nevertheless, a single gene was shown to have significant expression only in schizophrenia derived hiPSC: gene ZNF560, expressing the zinc finger protein 560.

Also, additional available data at publicly available genomic repositories, show a significant differential expression of the ZNF560 gene between healthy control and schizophrenia hiPSC (FIG. 1A). The ROC curve of ZNF560 expression data shows an AUC of 0.9 which classifies this parameter as an excellent discriminator of healthy vs schizophrenia conditions (FIG. 1B).

It should be noted that the ROC curves indicate a cutoff of 21.96 TPM; above that value indicates 100% specificity and 71% sensitivity for classification in the schizophrenia group.

ZNF560 belongs to the Kroppel-associated box (KRAB) domain-containing zinc finger proteins (KZFP) family, a large family of nucleic acid-binding proteins, with 378 members in the human genome. KZFPs bind to specific DNA regions and recruits KRAB-associated protein 1 (KAP1), which mediates the interaction of several epigenetic modifiers resulting in gene silencing (de Tribolet-Hardy 2024). Therefore, the specific expression of ZNF560 in schizophrenia may lead to aberrant epigenetic silencing that could be implicated in the phenotypes observed in these cells.

### Example 2: Individual Determination of Gene Overexpression of ZNF560 Between Reprogrammed Fibroblasts and Respective Isogenic hiSPC from Healthy Subjects and Schizophrenia Conditions

### Fibroblast Cell Lines:

Fibroblasts from healthy controls and schizophrenia patients were donated from Instituto D'Or for Research and Education, Brazil. Skin biopsies were collected and directly plated in DMEM high glucose medium with 1% penicillin-streptomycin (Thermo Fisher Scientific, USA). Human fibroblasts spontaneously migrated after two weeks and were expanded in standard culture conditions (37° C. in 5% CO2) for 3 passages. The cells were tested for Mycoplasma (MycoAlert™ PLUS, Lonza, USA) before any further manipulation. Cells at passage 3 were used for iPSC generation (Sochacki 2016).

Expansion of Fibroblasts was performed in DMEM high glucose supplemented with 10% Fetal Bovine Serum.

hiPSC Reprogramming:

Protocol for cell reprogramming and characterization of these cells has been previously published (Sochacki 2016). Briefly, cells were reprogrammed using the integration-free CytoTune®-iPS 2.0 Sendai Reprogramming Kit (Thermo Fisher Scientific, USA), which contains Sendai virus particles of the four Yamanaka factors (Takahashi et al., 2007). Briefly, 3.0-3.5×105 human dermal fibroblasts were plated onto one well of a gelatin-coated 6-well plate 24 h before virus transduction. The cells were transduced according to the manufacturer's protocol. Four days after transduction, cells were plated onto MEF feeder cells and fed with iPS cells medium supplemented with 30 ng/mL freshly added bFGF (Thermo Fisher Scientific, USA).

About 7 days after plating, the first appearing colonies were picked for expansion into individual hiPSC lines and transferred onto Matrigel (BD Biosciences, USA)-coated plates and cultured with E8 medium (Thermo Fisher Scientific, USA). After successful adaptation to feeder-free conditions of at least 3 iPSC colonies from each patient, the cells were expanded in E8 medium using 0.5 mM EDTA passaging solution (both from Thermo Fisher Scientific, USA).

Further cell expansion was performed in mTESR1 medium (StemCells Technologies Cat Nom 85850, Canada).

### RNA Extraction:

Cells were homogenized in Trizol and then total RNA was extracted using Direct-zol RNA MiniPrep (Zymo Research Cat No R2052, Irvine, CA).

### RT-qPCR:

cDNA was synthesized from 1 μg of total RNA using M-MuLV Reverse Transcriptase (NewEngland Biolabs, M0253S, Ontario, Canada). Relative expression of ZNF560 mRNA was assessed by qPCR using SyberGreen II mix (Agilent Technologies Thermocycler, Santa Clara, CA, USA) and the following primers:

Forward: SEQ ID NO: 3; and Reverse: SEQ ID NO 4.

mRNA Sequence of ZNF560:
>NM_152476.3 Homo sapiens zinc finger protein 560 (ZNF560), transcript variant 1, mRNA: SEQ ID NO: 1
>NM_001351678.2 Homo sapiens zinc finger protein 560 (ZNF560), transcript variant 2, mRNA: SEQ ID NO: 2.

### Results:

The evaluation of ZNF560 mRNA expression in hiPSC and the isogenic fibroblast that were reprogrammed, indicates that ZNF560 is expressed in control fibroblast and then is silenced upon reprogramming (FIG. 1C).

### Example 3: Analysis of DNA Methylation at the CpG Island Located in the Promoter Region of the ZNF560 Gene

### Methylation Sequencing of ZNF560 CpG Island:

Genomic DNA was extracted using Quick-DNA MiniPrep (Zymo Research Cat. No D3024, Irvine, CA). To identify methylation of 5mC in DNA samples, 500 ng of DNA was bisulfite-converted using EZ DNA Methylation-Gold Kit (Zymo Research Cat. No D5005, Irvine, CA).

The CpG island located at the promoter of ZNF560 was amplified from the Bisulfite-converted DNA by PCR.

### PCR Protocol:

94° C. for 3 min

40 cycles of 94° C. for 5 sec, 55° C. for 30 sec, 72° C. for 40 sec

72° C. for 10 min.

The sequence of CpG island is shown in SEQ ID NO: 5.

Forward (5′-3′) primer is shown in SEQ ID NO: 6; and reverse (3′-5′) primer is shown in SEQ ID NO: 7.

The PCR product was purified and cloned with a commercial TOPO TA Cloning Kit (Invitrogen Cat No: 450641, Carlsbad, CA). Sequences were obtained by Sanger method using M13 primers. Sequences were analyzed using BiQ Analyzer (Bock 2005).

### Results:

The analysis of DNA methylation at the CpG island located in the promoter region of the ZNF560 gene, shows significantly reduced methylation (1.6% methylation) in fibroblasts and that this value raises up to 91% methylation in healthy control hiPSC after reprogramming. On the contrary, only about 57% of the island becomes methylated in schizophrenia hiPSC, mechanistically supporting the persistence of ZNF560 expression upon reprogramming of these schizophrenia patient cells (FIG. 1D). This data has led the inventors to conclude the existence of “reprogramming-resistant” genes (e.g. ZNF560) that can represent markers for this disease.

The implications of the use of new technologies, such as hiPSC modeling, to study psychiatric disorders has gained support in biomedicine in recent years. The possibility that gene regulators could persist from patient tissue to reprogrammed cells adds a new complexity layer in the field. Hence, the inventors consider that ZNF560 expression measurements in schizophrenia-derived hiPSC represents a novel disease marker.

### Example 4: Validation of the Determination of the Gene Expression of ZNF560 Through RNA-Seq

The determination of the gene expression of ZNF560 by RNA-seq carried out in Example 1 was validated using all the public RNA-seq data available that had been performed on hiPSC obtained from healthy subjects (HC) and patients with schizophrenia (SZ). The databases included GEO, SRA and ENA.

Only samples reprogrammed from dermal fibroblasts were considered, regardless of the reprogramming method used. As an internal control of an effective reprogramming, only those datasets that had reads of the pluripotency genes OCT4 (POU5F1) and NANOG greater than 1 TPM were selected. In total, 99 RNA-seq from hiPSC HC and 7 RNA-seq from hiPSC SZ are included.

### Datasets:

TABLE 1
Datasets used for RNA-seq validation.

| Cell/Dataset identification | Diagnostic |
|---|---|
| Coriell Institute NoGM23761 | Schizophrenia* |
| Coriell Institute NoGM23760 | Schizophrenia* |
| EZQ3 (Sochacki et al., 2016) | Schizophrenia* |
| EZQ4 (Sochacki et al., 2016) | Schizophrenia* |
| SRX13198487 | Schizophrenia |
| SRX13198355 | Schizophrenia |
| SRX13198518 | Schizophrenia |
| Coriell Institute NoGM23279 | Healthy Control* |
| CF1 (Casas et al., 2018) | Healthy Control* |
| CF2 (Casas et al., 2018) | Healthy Control* |
| Control 37L25 (Zhang et al., | Healthy Control* |
| 2013) | |
| Control (Almeida et al., 2012) | Healthy Control* |
| Control 3 (Biswas et al., 2016) | Healthy Control* |
| SRX13198513 | Healthy Control |
| SRX13198294 | Healthy Control |
| SRX13198299 | Healthy Control |
| GSM2072610 | Healthy Control |
| GSM2072609 | Healthy Control |
| SRR5576291 | Healthy Control |
| ERZ267062 | Healthy Control |
| ERZ267021 | Healthy Control |
| ERZ267057 | Healthy Control |
| ERZ266973 | Healthy Control |
| ERZ487572 | Healthy Control |
| ERZ487610 | Healthy Control |
| ERZ376095 | Healthy Control |
| ERZ376154 | Healthy Control |
| ERZ376098 | Healthy Control |
| ERZ266997 | Healthy Control |
| ERZ267022 | Healthy Control |
| ERZ266976 | Healthy Control |
| ERZ267013 | Healthy Control |
| ERZ266983 | Healthy Control |
| ERZ487639 | Healthy Control |
| ERZ267048 | Healthy Control |
| ERZ267036 | Healthy Control |
| ERZ267025 | Healthy Control |
| ERZ487558 | Healthy Control |
| ERZ376132 | Healthy Control |
| ERZ376166 | Healthy Control |
| ERZ266985 | Healthy Control |
| ERZ267063 | Healthy Control |
| ERZ376119 | Healthy Control |
| ERZ266998 | Healthy Control |
| ERZ376128 | Healthy Control |
| ERZ487601 | Healthy Control |
| ERZ487590 | Healthy Control |
| ERZ487594 | Healthy Control |
| ERZ376037 | Healthy Control |
| ERZ376162 | Healthy Control |
| ERZ266994 | Healthy Control |
| ERZ376115 | Healthy Control |
| ERZ267024 | Healthy Control |
| ERZ376051 | Healthy Control |
| ERZ376146 | Healthy Control |
| ERZ376039 | Healthy Control |
| ERZ266980 | Healthy Control |
| ERZ267061 | Healthy Control |
| ERZ376036 | Healthy Control |
| ERZ376116 | Healthy Control |
| ERZ267009 | Healthy Control |
| ERZ487644 | Healthy Control |
| ERZ267064 | Healthy Control |
| ERZ267018 | Healthy Control |
| ERZ376148 | Healthy Control |
| ERZ376147 | Healthy Control |
| ERZ487568 | Healthy Control |
| ERZ376032 | Healthy Control |
| ERZ376048 | Healthy Control |
| ERZ376045 | Healthy Control |
| ERZ487611 | Healthy Control |
| ERZ487642 | Healthy Control |
| ERZ487625 | Healthy Control |
| ERZ376133 | Healthy Control |
| ERZ376117 | Healthy Control |
| ERZ376058 | Healthy Control |
| ERZ487600 | Healthy Control |
| ERZ376040 | Healthy Control |
| ERZ376089 | Healthy Control |
| ERZ267002 | Healthy Control |
| ERZ376082 | Healthy Control |
| ERZ267032 | Healthy Control |
| ERZ266995 | Healthy Control |
| ERZ266989 | Healthy Control |
| ERZ487634 | Healthy Control |
| ERZ376139 | Healthy Control |
| ERZ376102 | Healthy Control |
| ERZ487578 | Healthy Control |
| ERZ267054 | Healthy Control |
| ERZ266999 | Healthy Control |
| ERZ376136 | Healthy Control |
| ERZ376138 | Healthy Control |
| ERZ376092 | Healthy Control |
| ERZ376104 | Healthy Control |
| ERZ266974 | Healthy Control |
| ERZ267023 | Healthy Control |
| ERZ267033 | Healthy Control |
| ERZ267053 | Healthy Control |
| ERZ267055 | Healthy Control |
| ERZ376073 | Healthy Control |
| ERZ376055 | Healthy Control |
| ERZ376110 | Healthy Control |
| ERZ267066 | Healthy Control |
| ERZ267004 | Healthy Control |
| ERZ376152 | Healthy Control |
| ERZ487606 | Healthy Control |

*RNA-seq experiments from the present inventors for this technology.

### Results:

The graph of FIG. 2A shows all expression data (RNA-seq) obtained from public sources and from the experiments from the present inventors, indicating a significant differential expression of the ZNF560 gene between healthy control and schizophrenia hiPSC, which is comparable with the results shown in FIG. 1A of Example 1.

To evaluate the performance of ZNF560 as a diagnostic test able to discriminate between Control and SZ-hiPSC, a ROC curve of the data shown in FIG. 2A was performed. ROC-AUC is 0.92, confirming that ZNF560 is a good discriminator (see FIG. 2B).

Since only a few datasets from SZ-iPSC were found to meet the pluripotency requirement (NANOG and POU5F1>1 TPM), the data is unbalanced. A Precision-Recall curve (PR) was generated, which has better performance in evaluating unbalanced data. PR-AUC is 0.78, which is 10 times higher than the baseline (0.071), which confirms ZNF560 as a good discriminator between HC and SZ-iPSC (see FIG. 2C).

### Example 4: Validation of the Determination of the Gene Expression of ZNF560 Through RT-qPCR

The measurement of ZNF560 mRNA levels by RT-qPCR carried out in Example 2 was validated in two new hiPSC HC samples and two hiPSC SZ samples.

TABLE 2
Datasets used for RT-qPCR validation

| Identification | Diagnosis | Code and references |
|---|---|---|
| Control 1 | Healthy control | Coriell Institute NoGM23279 |
| Control 2 | Healthy control | CF1 (Casas et al., 2018) |
| Control 3 | Healthy control | CF2 (Casas et al., 2018) |
| Control 4 | Healthy control | Control 37L25 (Zhang et al., |
| | | 2013) |
| Control 5 | Healthy control | Control (Almeida et al., 2012) |
| Control 6 | Healthy control | Gibco ™ episomal hiPSC line |
| | | A18945 |
| SZ1 | Schizophrenia | Coriell Institute NoGM23761 |
| SZ2 | Schizophrenia | Coriell Institute NoGM23760 |
| SZ3 | Schizophrenia | Coriell Institute NoGM23762 |
| SZ4 | Schizophrenia | EZQ3 (Sochacki et al., 2016) |
| SZ5 | Schizophrenia | EZQ4 (Sochacki et al., 2016) |
| SZ6 | Schizophrenia | EZQ9 (Sochacki et al., 2016) |

In hiPSC SZ samples, ZNF560 appears in cycles lower than 30 Ct, when the values of the housekeeping gene GAPDH are lower than 18 cycles. The relative expression of ZNF560 is at least 50 times higher when comparing the hiPSC HC sample with the highest expression to the hiPSC SZ sample with the lowest expression.

The graph of FIG. 3 shows the ZNF560 mRNA levels relative to the expression of one hiPSC SZ sample, confirming that there is a higher expression of ZNF560 in the latter samples in comparison to the ones derived from healthy subjects. The data are normalized to the expression of GAPDH. N=6 HC, N=6 SZ. Therefore, from these results it is possible to conclude that the detection of ZNF560 transcript in a sample would correspond to a patient having schizophrenia, in comparison to the control sample, which has undetectable levels of ZNF560 transcript.

### REFERENCES

Almeida, S., Zhang, Z., Coppola, G., Mao, W., Futai, K., Karydas, A., Geschwind, M. D., Tartaglia, M. C., Gao, F., Gianni, D., Sena-Esteves, M., Geschwind, D. H., Miller, B. L., Farese Jr., R. V, & Gao, F.-B. (2012). Induced Pluripotent Stem Cell Models of Progranulin-Deficient Frontotemporal Dementia Uncover Specific Reversible Neuronal Defects. Cell Reports, 2(4), 789-798. https://doi.org/10.1016/j.celrep.2012.09.007
Bimbaum, R., & Weinberger, D. R. (2017). Genetic insights into the neurodevelopmental origins of schizophrenia. Nature reviews. Neuroscience, 18(12), 727-740. https://doi.org/10.1038/nrn.2017.125
Bock, C., Reither, S., Mikeska, T., Paulsen, M., Walter, J., & Lengauer, T. (2005). BiQ Analyzer: visualization and quality control for DNA methylation data from bisulfite sequencing. Bioinformatics (Oxford, England), 21(21), 4067-4068. https://doi.org/10.1093/bioinformatics/bti652
Casas, B. S., Vitória, G., do Costa, M. N., Madeiro da Costa, R., Trindade, P., Maciel, R., Navarrete, N., Rehen, S. K., & Palma, V. (2018). hiPSC-derived neural stem cells from patients with schizophrenia induce an impaired angiogenesis. Translational psychiatry, 8(1), 48. https://doi.org/10.1038/s41398-018-0095-9
Das, D., Feuer, K., Wahbeh, M., & Avramopoulos, D. (2020). Modeling Psychiatric Disorder Biology with Stem Cells. Current psychiatry reports, 22(5), 24. https://doi.org/10.1007/s11920-020-01148-1
de Tribolet-Hardy, J., Thorball, C. W., Forey, R., Planet, E., Duc, J., Coudray, A., Khubieh, B., Offner, S., Pulver, C., Fellay, J., Imbeault, M., Turelli, P., & Trono, D. (2023). Genetic features and genomic targets of human KRAB-zinc finger proteins. Genome research, 33(8), 1409-1423. https://doi.org/10.1101/gr.277722.123
Harrison, G., Hopper, K., Craig, T., Laska, E., Siegel, C., Wanderling, J., Dube, K. C., Ganev, K., Giel, R., an der Heiden, W., Holmberg, S. K., Janca, A., Lee, P. W., León, C. A., Malhotra, S., Marsella, A. J., Nakane, Y, Sartorius, N., Shen, Y, Skoda, C., . . . Wiersma, D. (2001). Recovery from psychotic illness: a 15- and 25-year international follow-up study. The British journal of psychiatry: the journal of mental science, 178, 506-517. https://doi.org/10.1192/bjp.178.6.506
Hoffman, G. E., Schrode, N., Flaherty, E., & Brennand, K. J. (2019). New considerations for hiPSC-based models of neuropsychiatric disorders. Molecular psychiatry, 24(1), 49-66. https://doi.org/10.1038/s41380-018-0029-1
Kadakia, A., Catillon, M., Fan, Q., Williams, G. R., Marden, J. R., Anderson, A., Kirson, N., & Dembek, C. (2022). The Economic Burden of Schizophrenia in the United States. The Journal of clinical psychiatry, 83(6), 22m14458. https://doi.org/10.4088/JCP.22m14458
Karagiannis, P., Takahashi, K., Saito, M., Yoshida, Y., Okita, K., Watanabe, A., Inoue, H., Yamashita, J. K., Todani, M., Nakagawa, M., Osawa, M., Yashiro, Y., Yamanaka, S., & Osafune, K. (2019). Induced Pluripotent Stem Cells and Their Use in Human Models of Disease and Development. Physiological reviews, 99(1), 79-114. https://doi.org/10.1152/physrev.00039.2017
Organización Panamericana de la Salud. (2023). Informe mundial sobre la salud mental: Transformar la salud mental para todos. OPS. https://doi.org/10.37774/9789275327715Singh, T., Poterba, T., Curtis, D., Akil, H., Al Eissa, M., Barchas, J. D., Bass, N., Bigdeli, T. B., Breen, G., Bromet, E. J., Buckley, P. F., Bunney, W. E., Bybjerg-Grauholm, J., Byerley, W. F., Chapman, S. B., Chen, W. J., Churchhouse, C., Craddock, N., Cusick, C. M., DeLisi, L., . . . Daly, M. J. (2022). Rare coding variants in ten genes confer substantial risk for schizophrenia. Nature, 604(7906), 509-516. https://doi.org/10.1038/s41586-022-04556-w
Sochacki, J., Devalle, S., Reis, M., de Moraes Maciel, R., da Silveira Paulsen, B., Brentani, H., Belmonte-de-Abreu, P. S., & Rehen, S. (2016). Generation of iPS cell lines from schizophrenia patients using a non-integrative method. Stem cell research, 17(1), 97-101. https://doi.org/10.1016/j.scr.2016.05.017
Stepnicki, P., Kondej, M., & Kaczor, A. A. (2018). Current Concepts and Treatments of Schizophrenia. Molecules (Basel, Switzerland), 23(8), 2087. https://doi.org/10.3390/molecules23082087
Richetto, J., & Meyer, U. (2021). Epigenetic Modifications in Schizophrenia and Related Disorders: Molecular Scars of Environmental Exposures and Source of Phenotypic Variability. Biological psychiatry, 89(3), 215-226. https://doi.org/10.1016/j.biopsych.2020.03.008
Takahashi, K., Tanabe, K., Ohnuki, M., Narita, M., Ichisaka, T., Tomoda, K., & Yamanaka, S. (2007). Induction of pluripotent stem cells from adult human fibroblasts by defined factors. Cell, 131(5), 861-872. https://doi.org/10.1016/j.cell.2007.11.019
Trokovic R, Weltner J, Nishimura K, Ohtaka M, Nakanishi M, Salomaa V, Jalanko A, Otonkoski T, Kyttala A. Advanced feeder-free generation of induced pluripotent stem cells directly from blood cells. Stem Cells Transl Med. 2014 December; 3(12):1402-9. doi: 10.5966/sctm.2014-0113. Epub 2014 Oct. 29. PMID: 25355732; PMCID: PMC4250212.
Zhang, Z., Almeida, S., Lu, Y., Nishimura, A. L., Peng, L., Sun, D., Wu, B., Karydas, A. M., Tartaglia, M. C., Fong, J. C., Miller, B. L., Farese Jr, R. V, Moore, M. J., Shaw, C. E., & Gao, F.-B. (2013). Downregulation of MicroRNA-9 in iPSC-Derived Neurons of FTD/ALS Patients with TDP-43 Mutations. PLOS ONE, 8(10), e76055-. https://doi.org/10.1371/journal.pone.0076055
Zhou, T., Benda, C., Duzinger, S., Huang, Y., Li, X., Li, Y., Guo, X., Cao, G., Chen, S., Hao, L., Chan, Y. C., Ng, K. M., Ho, J. C., Wieser, M., Wu, J., Redl, H., Tse, H. F., Grillari, J., Grillari-Voglauer, R., Pei, D., . . . Esteban, M. A. (2011). Generation of induced pluripotent stem cells from urine. Journal of the American Society of Nephrology: JASN, 22(7), 1221-1228. https://doi.org/10.1681/ASN.2011010106

What is claimed is:

1. A mix of primers capable of amplifying a biomarker associated with gene ZNF560, wherein the forward primer comprises a nucleotide sequence having at least an 80%, 90% or 95% of identity of SEQ ID NO: 3 and the reverse primer comprises a nucleotide sequence having at least an 80%, 90% or 95% of identity of SEQ ID NO: 4.

2. The mix of primers capable of amplifying a schizophrenia biomarker associated with gene ZNF560 of claim 1, wherein the forward primer comprises a nucleotide sequence comprising SEQ ID NO: 3 and the reverse primer comprises a nucleotide sequence comprising SEQ ID NO: 4.

3. A kit of parts to diagnose schizophrenia from a biological sample, comprising:
a) a leaflet with instructions to develop hiPSC from a biological sample and to perform the technique to measure of the gene expression of ZNF560, wherein said technique is RNA-seq or a RT-qPCR; and
b) a mix of primers if the technique is RT-qPCR, wherein these primers are any appropriate primers to generate a cDNA of gene ZNF560, and wherein the primers are the primers of claim 1.

4. A kit of parts to diagnose schizophrenia from a biological sample, comprising:
a) a leaflet with instructions to develop hiPSC from a biological sample and to perform the technique to measure of the gene expression of ZNF560, wherein said technique is RNA-seq or a RT-qPCR; and
b) a mix of primers if the technique is RT-qPCR, wherein these primers are any appropriate primers to generate a cDNA of gene ZNF560.

5. The kit of claim 4, wherein the kit comprises optionally the required reagents to perform a RT-qPCR, such as a fluorescent dye, a DNA polymerase, a Reverse transcriptase, dNTPs, a magnesium salt, probes and any combination thereof.

6. The kit of claim 5, wherein the magnesium salt is selected from MgCl₂, MgSO₄ and Mg(OAc)₂.


SEQUENCE LISTING (7 sequences)

SEQ ID NO: 1 - DNA, 2815 bp, Homo sapiens
  source 1..2815: mol_type = genomic DNA
agttctctcttttggtcgctggcgcctgtttcctcagagccactttttcctttgtgttgtctggtcgttcactactttgagggaaaagaattttgccgttcgcgttcgattgactccgtccccaagatcctgtagccccacggacctggcgactccaccgtgttcgggtctttctaggaacccaatctgccttcatgaagaaaagacagaggcagaaaggaagatggcttactgcctgacaaattgttatcagtactccgtgacctttgaggatacagctgtggacttcacccaggaggagtggattttactggacccagttcagagaaacttatacagagatgtgatgctggagaattatgagaacgtggccaaagtaggatttcagctctttaaacccagtgtcatctcttggttggaagaagaagagttgagaaccttgcagcaaggagttctccaagactgggcaataaaacatcaaaccagtgtttcagcactgcagcaggagttttggaaaatacaaacttctaatgggatacaaatggacctggtaacctttgacagtgtggctgtggagttcacccaggaagagtggactttactggacccagctcagagaaacctgtacagtgatgtgatgctggagaactacaagaacctgtcctcagtaggttaccagctcttcaaacccagtctgatctcttggctggaggaagaggaagagttgagcacactgccaagagttctccaagaatggaaaatgtgcctgaaaaccaaagggccagccctttggcaagataatttttgtttaaaaacattaaatgggatacagttggcaagaaaccaaaatggagaggaactctatgactgtaagcaatgtgaagatgtcttctgtaaacatccatgccttaagaccaacatgagtactcaaaatagaggcaacacgtctgaatgtattcagtatgcaaaagacctcctttctctatacaataaaacctctaccataaggaaagtttctgtgttcagtaagcatggaaaatctttccgcctgattttaaatgttcaggtccagagaaagtgtacacaagataaatcctttgaaggcactgactatgggaaagccttcatttatcagtcataccttgaagcacacaggaaaactcagagtggagaaaaactcaatgaatggaagcaatgtggggaagcatttactcactccacaagccatgctgtaaatgttgaaacacacattattaaaaacccctatgaatgtaaggaatgtggaaaagattttagataccctacccaccttaataatcacatgcaaacccacattgggataaaaccttataaatgtaagcactgtggcaaaaccttcactgtgccgtcaggctttcttgaacatgtacgaactcacactggagagaagccctatgggtgtaaggaatgtggtaaagcctttggtacatctgcaggccttattgaacatataagatgtcacgccagagagaaaacctttaaatgtgaccactgtgggaaagcctttatttcttacccatctctttttggacatttgagagttcataatggagagaagccatatgagcataaggaatatgggaaggcctttggtacatcctcaggtgttattgaagatagaagaagtaacacaggacagaaacgctttgattgtgaccagtgtgggaaagtctttgtttctttctcatctctttttgctcatttgagaactcacactggtgagaagccctttaagtgttacaaatgtgggaaaccatttacctcttctgcctgtcttcgtattcacatgcgaactcacacagaagagagactctatcaatgtaagaaatgtggtaaagctttcactaagtgctcatatcttaccaaacatttacgaacacacgctggagagaaaccctatgaatgtatgaaatgtgggaaagccttcactgagcgctcataccttactaaacatttacgaagacacagtggagagaagccatatgaatgtaagaaatgtggaaaagccttcacagaacgctcagatcttactaaacatttacgaagacacactggagataagccctatgaatataaggactgtgggaaagcctttgttgtctcctccagtctagttgatcatttaagaactcacactggatataaaccctataaatgtaatgcatgtgaaaaagcttacagtaggtcttgtgtactaactcaacacttaaaaactcatgcagcagagaagacctctgaatgtaacgcatgtggaaattcctttcgaaattccatgtgctttcatgatcgcttaaaaactctcaccaaaataaaaccctataaatgtaaggactgtgggaaagccttcacttgtcattcagatcttactaatcatgtgcgaattcacactggagagaagccctataaatgtaaggaatgtgggaaggccttccgtacatcctcaggacgtattcaacatttaagaactcatatgggagagaaaccctttgaatgtgaccagtgtgggaaagcctttgcttctttctcagctcgtattgcacatttgaaaacacactagagagaagccctgtggatgtgaagaatgtggaaaaacctttgctgtttcctcaagcctaactgaacatgtaaaaattcacaggagaaagtaacactatcaatgtaaggaatgtgagaaagcctgaacagttgaatctttctagatatgtacaaactcacactgtagagaaaccctgtaaatgtaagggatgtaggaaaacactagttcttctctctctct

SEQ ID NO: 2 - DNA, 2811 bp, Homo sapiens
  source 1..2811: mol_type = genomic DNA
agttctctcttttggtcgctggcgcctgtttcctcagagccactttttcctttgtgttgtctggtcgttcactactttgagggaaaagaattttgccgttcgcgttcgattgactccgtccccaagatcctgtagccccacggacctggcgactccaccgtgttcgggtctttctaggaacccaatctgccttcatgaagaaaagacagaggcagaaaggaagatggcttactgcctgacaaattgttatcagtactccgtgacctttgaggatacagctgtggacttcacccaggaggagtggattttactggacccagttcagagaaacttatacagagatgtgatgctggagaattatgagaacgtggccaaagatttcagctctttaaacccagtgtcatctcttggttggaagaagaagagttgagaaccttgcagcaaggagttctccaagactgggcaataaaacatcaaaccagtgtttcagcactgcagcaggagttttggaaaatacaaacttctaatgggatacaaatggacctggtaacctttgacagtgtggctgtggagttcacccaggaagagtggactttactggacccagctcagagaaacctgtacagtgatgtgatgctggagaactacaagaacctgtcctcagtaggttaccagctcttcaaacccagtctgatctcttggctggaggaagaggaagagttgagcacactgccaagagttctccaagaatggaaaatgtgcctgaaaaccaaagggccagccctttggcaagataatttttgtttaaaaacattaaatgggatacagttggcaagaaaccaaaatggagaggaactctatgactgtaagcaatgtgaagatgtcttctgtaaacatccatgccttaagaccaacatgagtactcaaaatagaggcaacacgtctgaatgtattcagtatgcaaaagacctcctttctctatacaataaaacctctaccataaggaaagtttctgtgttcagtaagcatggaaaatctttccgcctgattttaaatgttcaggtccagagaaagtgtacacaagataaatcctttgaaggcactgactatgggaaagccttcatttatcagtcataccttgaagcacacaggaaaactcagagtggagaaaaactcaatgaatggaagcaatgtggggaagcatttactcactccacaagccatgctgtaaatgttgaaacacacattattaaaaacccctatgaatgtaaggaatgtggaaaagattttagataccctacccaccttaataatcacatgcaaacccacattgggataaaaccttataaatgtaagcactgtggcaaaaccttcactgtgccgtcaggctttcttgaacatgtacgaactcacactggagagaagccctatgggtgtaaggaatgtggtaaagcctttggtacatctgcaggccttattgaacatataagatgtcacgccagagagaaaacctttaaatgtgaccactgtgggaaagcctttatttcttacccatctctttttggacatttgagagttcataatggagagaagccatatgagcataaggaatatgggaaggcctttggtacatcctcaggtgttattgaagatagaagaagtaacacaggacagaaacgctttgattgtgaccagtgtgggaaagtctttgtttctttctcatctctttttgctcatttgagaactcacactggtgagaagccctttaagtgttacaaatgtgggaaaccatttacctcttctgcctgtcttcgtattcacatgcgaactcacacagaagagagactctatcaatgtaagaaatgtggtaaagctttcactaagtgctcatatcttaccaaacatttacgaacacacgctggagagaaaccctatgaatgtatgaaatgtgggaaagccttcactgagcgctcataccttactaaacatttacgaagacacagtggagagaagccatatgaatgtaagaaatgtggaaaagccttcacagaacgctcagatcttactaaacatttacgaagacacactggagataagccctatgaatataaggactgtgggaaagcctttgttgtctcctccagtctagttgatcatttaagaactcacactggatataaaccctataaatgtaatgcatgtgaaaaagcttacagtaggtcttgtgtactaactcaacacttaaaaactcatgcagcagagaagacctctgaatgtaacgcatgtggaaattcctttcgaaattccatgtgctttcatgatcgcttaaaaactctcaccaaaataaaaccctataaatgtaaggactgtgggaaagccttcacttgtcattcagatcttactaatcatgtgcgaattcacactggagagaagccctataaatgtaaggaatgtgggaaggccttccgtacatcctcaggacgtattcaacatttaagaactcatatgggagagaaaccctttgaatgtgaccagtgtgggaaagcctttgcttctttctcagctcgtattgcacatttgaaaacacactagagagaagccctgtggatgtgaagaatgtggaaaaacctttgctgtttcctcaagcctaactgaacatgtaaaaattcacaggagaaagtaacactatcaatgtaaggaatgtgagaaagcctgaacagttgaatctttctagatatgtacaaactcacactgtagagaaaccctgtaaatgtaagggatgtaggaaaacactagttcttctctctctct

SEQ ID NO: 3 - DNA, 19 bp, synthetic construct
  source 1..19: mol_type = other DNA
gggatacagttggcaagaa

SEQ ID NO: 4 - DNA, 20 bp, synthetic construct
  source 1..20: mol_type = other DNA
agaaaggaggtcttttgcat

SEQ ID NO: 5 - DNA, 537 bp, Homo sapiens
  source 1..537: mol_type = genomic DNA
acccgaacacggtggagtcgccaggtccgtggggctacaggatcttggggacggagtcaatcgaacgcgaacggcaaaattcttttccctctgaaacacaattcacaaacgttgccttggagacttattttttaaggcctcagaacgcttggacacttttgaagacgggaacacacggctgaaaacagccctcggacgcgcccatactgcacttcccacccggctcctctaggggaccgaaggtcccaatggcctcttcgacgtggcctcacccgatggctccgcgactcctgcggactcctgtggagtggtccaggcctctgtaaggaggttcctcgattaccctgggtcgattgtgacctctgccaccccccgaaacaccgcccaacccactagtccacacgagcctcaccaaagtagtgaacgaccagacaacacaaaggaaaaagtggctctgaggaaacaggcgccagcgaccaaaagagagaactggcgcgccgcaacgtcacttccgtttggggcattggaaggggcgtg

SEQ ID NO: 6 - DNA, 22 bp, synthetic construct
  source 1..22: mol_type = other DNA
ttgttttggagatttatttttt

SEQ ID NO: 7 - DNA, 20 bp, synthetic construct
  source 1..20: mol_type = other DNA
aatcctaaaaaattccatcc